(19) United States
(12) Patent Application Publication
(10) Pub. No.: US 2026/0314992 A1
(43) Pub. Date: Oct. 15, 2026

(54) DEVICE FOR MONITORING SUDDEN UNEXPECTED DEATH IN EPILEPSY

(71) Applicant: Neurava Inc., Indianapolis, IN (US)

(72) Inventors: Jay Vatsal SHAH, Brownsburg, IN (US); Vivek GANESH, Indianapolis, MD (US); Trevor Donald MEYER, Baltimore, MD (US); Juan Ernesto RODRIGUEZ, Woodside, CA (US); Dominic PERALTA, San Mateo, CA (US); Tonatiuh ESQUIVEL, Chico, CA (US)

(21) Appl. No.: 19/125,961

(22) PCT Filed: Oct. 31, 2023
(86) PCT No.: PCT/US2023/036450

§ 371 Date: Apr. 30, 2025

Related U.S. Application Data
(60) Provisional application No. 63/421,309, filed on Nov. 1, 2022.

(51) Int. Cl. A61B 5/00; A61B 5/0205; A61B 5/08; A61B 5/1455; A61B 5/346

(52) U.S. Cl. CPC A61B 5/4094; A61B 5/0205; A61B 5/08; A61B 5/14551; A61B 5/346; A61B 5/6814; A61B 5/6822; A61B 5/6823; A61B 5/6824; A61B 5/6828; A61B 5/746; A61B 2560/0219; A61B 2562/164

(57) ABSTRACT

A system for monitoring physiological parameters of a user for the detection of sudden unexpected death in epilepsy includes an ECG sensor, an SpO2 sensor, a respiration sensor, and a wearable housing sized and configured to be worn on a neck, a chest, an arm, a leg, a head, or a back of a user. The ECG sensor, the SpO2 sensor, and the respiration sensor are positioned in the wearable housing at locations configured to sense physiological parameters of the user associated with at least one of seizure activity, cardiorespiratory dysfunction, SUDEP prediction, SIDS prediction, and sleep apnea.

### CROSS-REFERENCE TO RELATED APPLICATION

This application claims the benefit of U.S. Provisional Application No. 63/421,309, filed on Nov. 1, 2022. The disclosure of the prior applications is hereby incorporated by reference in their entireties.

### TECHNICAL FIELD

This document relates to a medical device configured to be worn on the neck of a user and to monitor physiological parameters to detect seizure activity and cardiorespiratory dysfunctions for prevention of sudden unexpected death in epilepsy.

### BACKGROUND

Epilepsy, a brain disorder resulting in seizures, affects more than 65 million people worldwide. There are an estimated 3.5 million Americans-including 470,000 children-living with epilepsy. In approximately 35% of cases, seizures cannot be controlled by medication, and uncontrolled seizures are the primary risk factor for sudden, unexpected death from epilepsy (SUDEP). SUDEP is a sudden, unexpected, non-traumatic death, occurring in benign circumstances in an individual with epilepsy, with or without evidence that a seizure has occurred.

### SUMMARY

Some embodiments of a medical system include a wearable device having ECG, respiration, and SpO2 sensors within a wearable housing. The wearable housing can be sized and shaped to be worn on the neck of a user to monitor physiological parameters of the user. The monitored parameters can be used to determine occurrence of a seizure activity and/or other cardiorespiratory dysfunctions and to predict a likelihood of occurrence of a SUDEP event. The determination can occur within the wearable device, or can be made using software of an external client device, external processor or cloud-based server.

In a first aspect, a system includes an ECG sensor, an SpO2 sensor, a respiration sensor, and a wearable housing. The wearable housing is sized and configured to be worn on either the neck or the chest of a user. The ECG sensor, SpO2 sensor, and respiration sensor are positioned in the wearable housing at locations configured to sense at the neck or the chest of the user.

In another aspect, a system includes a housing sized and configured to be worn on a neck of a user, the housing having a first sensor node, a second sensor node, and a bridge extending between the first and second sensor nodes. A first set of one or more sensors is positioned in the first sensor node, and a second set of one or more sensors is positioned in the second sensor node.

In another aspect, a method includes positioning a sensor system on the neck of a patient, where the sensor system is configured to align an SpO2 sensor near a carotid artery of the patient, and to align first and second sensors with opposing sides of the neck of the patient. The method also includes outputting a sensor signal from the SpO2 and first and second sensors to an external device, where the signal includes respiration data, ECG data, and SpO2 data. The method also includes outputting a SUDEP determination from the external device based on the sensor signal.

In another aspect, a system includes a wearable device which includes an ECG sensor, an SpO2 sensor, a respiration sensor and a housing. The housing is sized and configured to be positioned on skin of a user, and the ECG sensor, the SpO2 sensor, and the respiration sensor are positioned on the wearable housing at locations configured to sense at the skin of the user. The system also includes a client device communicatively coupled to the wearable device, and a server communicatively coupled to the client device. The client device receives one or more signals from the wearable device, where the one or more signals include an ECG signal detected by the ECG sensor, an SpO2 signal detected by the SpO2 sensor, and a respiration signal detected by the respiration sensor. The client device also transmits data based on the ECG signal, SpO2 signal, and respiration signal to the server.

In another aspect, a system includes an ECG sensor, an SpO2 sensor, a respiration sensor, and a wearable housing. The wearable housing is sized and configured to be worn on one of an arm, a leg, a head, or a back of a user. Alternatively, the wearable housing can be sized and configured to be worn on one of an arm, a leg, a head, or a back of a user. The ECG sensor, SpO2 sensor, and respiration sensor are positioned in the wearable housing at locations configured to sense at the neck or the chest of the user.

Some or all of the embodiments described herein can have one or more of the following advantages. First, some embodiments of the medical device provide sensors that allow multiple physiological signals to be measured concurrently. The concurrently monitored physiological signals can be provided as inputs to an algorithm for enhanced detection of seizure activity and/or cardiorespiratory dysfunctions based on multiple continuously monitored physiological signals. The inputs to the algorithm can improve the detection of seizure activity and/or cardiorespiratory dysfunctions and prediction of SUDEP events based on the continuous data representing multiple different parameters from which patterns and changes can be extracted.

Second, some embodiments of the medical device measure multiple physiological parameters using a limited number of electrodes or sensors. For example, a pair of electrodes are used to monitor both respiration and ECG. The measurement of multiple parameters at a limited number of electrodes can minimize or reduce the size of the medical device, providing a smaller and more comfortable medical device compared to a device in which each parameter is measured using separate sensors or electrodes. Third, some embodiments of the medical device provide a comfortable and easy to position device that can be placed by a user without the help of a clinician or caregiver. For example, the medical device includes two nodes connected by a narrower bridge portion, and the two nodes are attached to the user's skin using medical-grade adhesive patches. The narrow bridge portion can provide flexibility between the two nodes, and can include a marker for placement at or near a midline of the user's neck. The marker can enable the user to properly position the medical device for accurate sensing of physiological parameters, for example by positioning electrodes at the first node and second node over the carotid arteries. Additionally, the marker and flexible bridge portion without adhesive can comfortably extend over the front part of the user's throat at the vocal cords and laryngeal prominence of thyroid cartilage allowing for comfortable breathing, swallowing, and talking while the medical device is in place.

Fourth, some embodiments of the medical device provide multiple sensors on a wearable device for detection of a broad range of seizure activity, including generalized tonic-clonic seizures and others. The multiple sensors and capability of the sensors to monitor multiple physiological parameters of the wearer can provide more accurate seizure detection, and can provide monitoring of other conditions. For example, some embodiments of the medical device can be used to monitor conditions including shock and sleep apnea, or can be used in monitoring vital signs. The data collected by the medical device during monitoring can be used in seizure forecasting, SUDEP prevention, seizure prediction, and sleep score determinations, among other uses.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1C illustrate an exemplary wearable device for SUDEP detection.

FIG. 2 illustrates an exemplary system including a wearable device for SUDEP detection.

FIG. 3 illustrates an exemplary system diagram of a wearable device for SUDEP detection.

FIG. 4 illustrates an exemplary positioning diagram for a wearable device positioned on a user's neck.

FIGS. 5A and 5B illustrate views of an exemplary wearable device.

FIGS. 6A-6C illustrate exemplary printed circuit boards for a wearable device.

FIGS. 7A-7D illustrate signals detected at a wearable device.

FIG. 8 illustrates an exemplary flow chart for a method of monitoring physiological parameters at a wearable device.

FIG. 9 illustrates an exemplary flow chart for a method of detecting seizure activity and cardiorespiratory dysfunctions before, during, and after seizure activity in a system including a wearable device.

FIG. 10 illustrates an exemplary flow chart for a method of monitoring seizure activity and cardiorespiratory dysfunctions before, during, and after seizure activity at a wearable device to predict a SUDEP incident.

FIG. 11 illustrates sensor outputs from an exemplary wearable device.

FIGS. 12A and 12B illustrate an alternative embodiment of an exemplary wearable device for SUDEP detection having a securement portion.

Like reference symbols in the various drawings indicate like elements.

### DETAILED DESCRIPTION OF ILLUSTRATIVE EMBODIMENTS

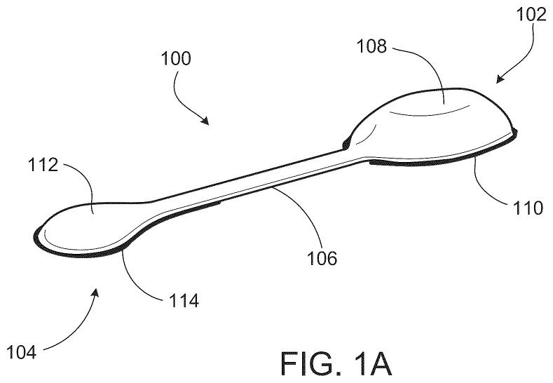

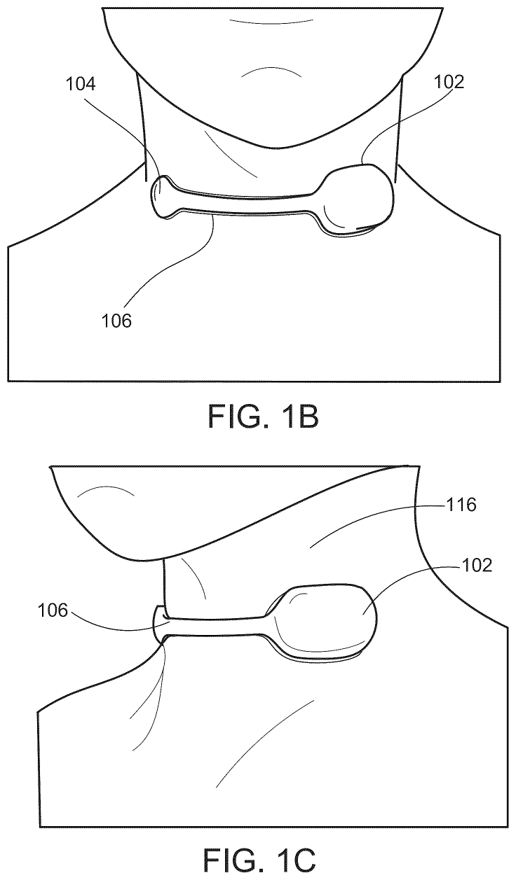

Referring to FIGS. 1A-1C, an example embodiment of a wearable device 100 includes a first node 102 and a second node 104 (the first node and second node are also referred to as “first end” and “second end” as well as “sensor nodes” herein). The first node 102 includes a first outer housing 108 and the second node 104 includes a second outer housing 112. The first outer housing 108 and the second outer housing 112 are coupled by a bridge portion 106. The wearable device 100 also includes a first adhesive 110 positioned on a bottom surface of the first node 102 opposite the first outer housing 108, and a second adhesive 114 positioned on a bottom surface of the second node 104 opposite the second outer housing 112.

The first adhesive 110 and the second adhesive 114 allow the wearable device 100 to be attached to a user's skin at a neck 116 of the user, as illustrated in FIGS. 1B and 1C. The first adhesive 110 and the second adhesive 114 can be reusable adhesives which can be used to attach the wearable device 100 to the neck 116 and to be removed and reattached multiple times. The first node 102 and the second node 104 can be attached to either side of the user's neck 116 with the bridge portion 106 extending over the midline of the neck 116. In some implementations, the first node 102 is positioned on the left side of the user's neck 116. In other implementations, the first node 102 is positioned on the right side of the user's neck 116. In some implementations, each of the first outer housing 108 and the second outer housing 112 are positioned on the neck 116 to overlay the left and right carotid arteries of the user. The bridge portion 106 is narrower in width and in height than the first outer housing 108 and the second outer housing 112, and is formed from a flexible material to provide bending of the wearable device 100 at the bridge portion 106. While the first outer housing 108 and second outer housing 112 are described here as separate housings, in some embodiments, the device housing can be formed as a modular housing or as a single housing component which contains the circuitry, components, and power source The wearable device 100 can include components to monitor seizure activity and any cardiorespiratory dysfunction (which can include monitoring physiological parameters associated with seizure activity, such as physiological parameters occurring before a seizure, during a seizure, or after a seizure) through the measurement of physiological parameters including cardiac and respiratory functions. As used herein, cardiorespiratory dysfunction refers to cardiac dysfunction or respiratory dysfunction, occurring together or individually. An indication of cardiorespiratory dysfunction can be an indication of a change in pattern in cardiac activity, breathing or other respiratory activity, or both. In some implementations, the wearable device 100 can also include components for processing the monitored physiologic parameters to determine seizure activity and any cardiorespiratory dysfunctions prior to SUDEP, and to determine a likelihood of SUDEP based at least in part on the detected seizure activity and the cardiorespiratory dysfunctions. In some implementations, the wearable device 100 can monitor physiological parameters and determine the incidence of multiple types of seizure activities. In some implementations, the wearable device 100 is used in combination with one or more additional sensors and one or more external user devices.

In some implementations, the wearable device 100 can monitor activities other than seizure activities, including sleep apnea, sleep patterns, likelihood of SIDS occurrence, or other conditions. In some implementations, the sensors on the wearable device can be altered in number, type or position to provide monitoring for a particular condition. In some implementations, the size of the wearable device can be selected to fit a wearer of the wearable device; for example a small wearable device for use with an infant.

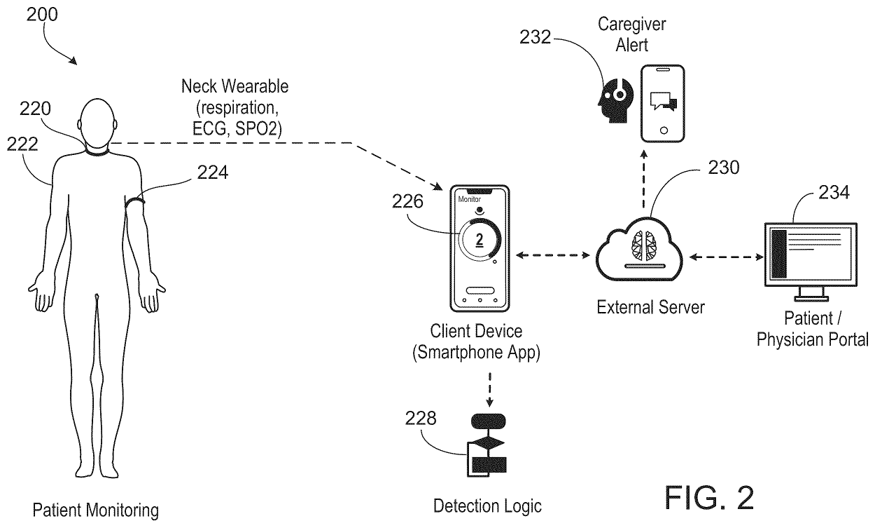

FIG. 2 illustrates an exemplary system 200 including a wearable device 220 for SUDEP detection. The system 200 includes a first wearable device 220 positioned on a user 222 for detection of physiological parameters. The system 200 further includes a client device 226 and an external processor 230. The first wearable device 220 (for example, wearable device 100 in FIGS. 1A-1C) monitors physiological parameters including respiration, ECG, and SpO2 of the user. The first wearable device 220 communicates a signal including the respiration, ECG, and SpO2 data to the client device 226. The client device 226 can be a computer, smart phone, tablet, or other client device capable of running a program such as a smartphone application. The client device 226 can include one or more processors and memory with instructions thereon for performing one or more functions, such as for detecting or predicting seizures and cardiorespiratory dysfunctions. The client device 226 includes detection logic 228 (e.g., seizure detection logic, SUDEP detection, prediction, and/or forecasting logic, and/or cardiorespiratory dysfunction detection logic) implemented in hardware and/or software. The client device 226 uses the seizure and/or the cardiorespiratory dysfunction detection logic 228 to process the signal including the respiration, ECG, and SpO2 data received from the wearable device 220, and determines whether seizure activity or any cardiorespiratory dysfunction is indicated by the data. For example, the detection logic 228 can analyze the data for changes, patterns, and events that indicate a seizure or cardiorespiratory disfunction has occurred or is likely to occur.

The client device 226 transmits information related to the respiration, ECG, and SpO2 data received from the wearable device 220 and the determination of seizure activity or any cardiorespiratory dysfunction by the seizure and/or the cardiorespiratory dysfunction detection logic 228 to an external processor 230. The external processor 230 can include one or more processors and memory with instructions thereon for performing one or more functions, such as for storing, processing, and analyzing data. The external processor 230 can include cloud-based storage, virtual machines, and external servers. The external processor 230 can complete additional processing and data analysis of the signal including the respiration, ECG, and SpO2data. In some embodiments, the detection logic 228 can be included in the external processor 230.

The external processor 230 transmits information related to the signal including the respiration, ECG, and SpO2 data and the determination of seizure activity or cardiorespiratory dysfunction by the detection logic 228 to a patient or physician portal 234. The patient or physician portal 234 can be implemented as a computer or as software used on a computer. In some implementations, the patient or physician portal 234 is part of an electronic health record. In some implementations, the patient or physician portal 234 is a secure website. The patient or physician portal 234 can be accessed by the user's doctor, clinician, or other medical professional to monitor the health and condition of the user. In some implementations, the patient or physician portal 234 can be accessed by the user or caregiver to monitor their health.

Additionally, the external processor 230 transmits information related to the signal including the respiration, ECG, and SpO2 data and the determination of seizure activity or the cardiorespiratory dysfunction by the seizure and/or the cardiorespiratory dysfunction detection logic 228 to a system for providing caregiver alerts 232. In some implementations, the system for providing caregiver alerts 232 is a call center, an application on an external device of a caregiver (e.g., a smart phone, tablet, or designated device), an alarm system, or an emergency medical center. The system for providing caregiver alerts 232 receives the information and, if seizure activity or the cardiorespiratory dysfunction is detected that may indicate a likelihood of SUDEP, the system for providing caregiver alerts 232 sends a message to a caregiver of the user to alert the caregiver so that they can intervene.

Typically a seizure is indicated by or preceded by changes in physiological parameters including the occurrence of bradycardia or tachycardia, irregular intervals of heart rate, and changes to breathing and respiratory patterns. The wearable device 220 monitors physiological parameters using sensors and processes the signals to determine whether a cardiorespiratory dysfunction has occurred, whether a seizure has occurred and/or whether a seizure or SUDEP event is likely to occur. In some implementations, one or more of the wearable device 220, the client device 226, and the external processor 230 uses machine learning or AI algorithms to analyze the detected signals to determine the likelihood of a SUDEP event.

In some implementations, only a subset of respiration, ECG, and SpO2 are monitored by the wearable device 220. In some implementations, additional physiological parameters are monitored at the wearable device 220 in addition to, or in the place of one or more of, the respiration, ECG, and SpO2. In some implementations, one or more additional sensor devices 224 positioned on the user 222 detect one or more physiological parameters or signals and transmit the signals to the client device 226.

In some implementations, the client device 226 communicates directly with the patient or physician portal and a caregiver device. In some implementations, the transmission of data and information to the patient or physician portal 234 is disabled. In some implementations, the transmission of data for caregiver alerts is disabled.

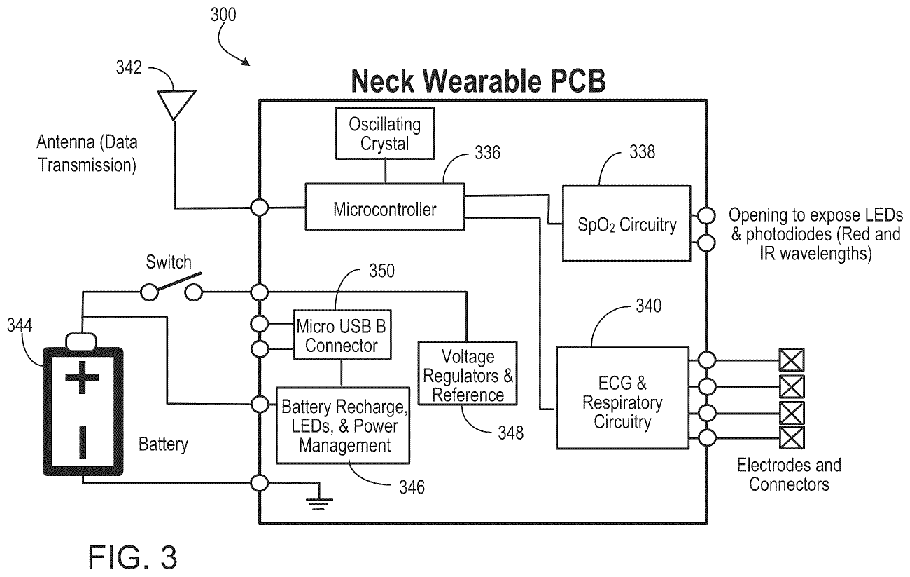

Referring to FIG. 3, a wearable device (for example, wearable device 100 of FIGS. 1A-1C, or wearable device 220 of FIG. 2) includes a microcontroller 336 (including one or more processors and memory with instructions thereon), SpO2 circuitry 338, ECG and respiration circuitry 340, a power management module 346, a voltage regulator 348, a data/power connector 350, battery 344, and an antenna 342.

The SpO2 circuitry 338 includes a sensor for determining SpO2. The sensor in the SpO2 circuitry is a pulse oximeter sensor capable of emitting light through LEDs and measuring a reflected wavelength of light directed toward the user's skin using photodetectors. In some implementations, the sensor incorporates red and infrared LEDs and photodetectors. In some implementations, the sensor is implemented in a single integrated circuit. The SpO2 percentage value is calculated using the measured reflectance values. The ECG and respiration circuitry 340 includes a sensor for detection of ECG using circuits and detection of respiration through measured bioimpedance. To detect the ECG, the sensor measures electrical activity at the user's skin. To detect the respiration, the ECG and respiration circuitry provides a current to the user's skin and measures a voltage change. The ECG and respiration circuitry 340 monitors both the ECG and respiration using a single electrode pair. The SpO2 circuitry 338 and the ECG and respiration circuitry 340 work in conjunction to monitor physiological parameters. In some implementations, the ECG and respiration circuitry 340 is implemented in a single integrated circuit.

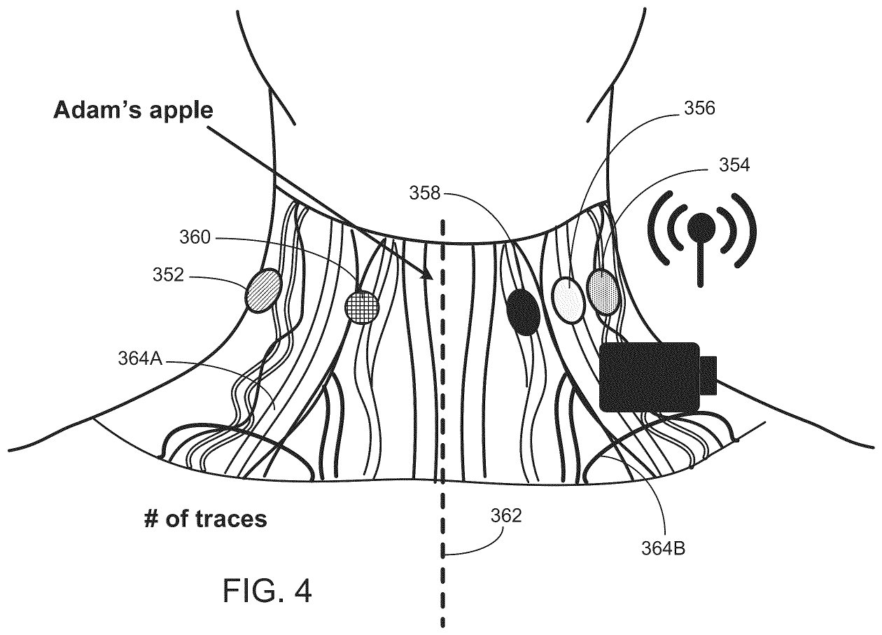

FIG. 4 illustrates an exemplary positioning of electrodes associated with the SpO2 circuitry 338 and the ECG and respiration circuitry 340 on a user's neck to enable monitoring of the SpO2, respiration, and ECG. The wearable device includes a first electrode 352, second electrode 354, third electrode 356, fourth electrode 358, and fifth electrode 360.

The first electrode 352, second electrode 354, and fourth electrode 358 correspond to the ECG and respiration circuitry 340 and are used in the detection of ECG and respiration data. The first electrode 352 and second electrode 354 are positioned on opposite sides of the neck, on either side of a midline 362 at the center of the neck. The first electrode 352 and second electrode 354 detect the voltage potential difference, and the fourth electrode 358 is a right-leg drive to improve signal quality by eliminating interference noise and improving the common mode rejection ratio. The first electrode 352 and second electrode 354 also monitor the bioimpedance to collect respiration data.

In some implementations, other types of sensors are included in the wearable device in addition or in the alternative to the sensors described above. In some implementations, a pressure sensor may be used to collect respiration data rather than through bioimpedance. In some implementations, the respiration signal may be derived from at least one of the ECG signal, red wavelength measured reflectance value, green wavelength measured reflectance value, IR wavelength measured reflectance value, and the SpO2 signal. In some implementations, an EMG sensor is included in the device for detection of muscle activity or detection of a laryngospasm. In some implementations, a microphone is included in the device for recording sounds associated with seizures (e.g., an ictal cry) or sounds associated with sleep apnea (e.g., snoring or gasping). In some implementations, a microphone and/or an EMG is included in the device for recording respiration.

The fifth electrode 360 is positioned near the right carotid artery 364A on the user's neck. The fifth electrode 360 includes at least one LED and a photodiode for transmitting and detecting red, green, and/or IR wavelengths of light to monitor the SpO2 in the blood of the user. The red, green, and/or IR measured reflectance values detected at the fifth electrode 360 can be converted to an SpO2 value. In some implementations, a third electrode 356 is positioned opposite the fifth electrode 360 across the midline 362 and over the left carotid artery 364B, though the third electrode 356 can be omitted from the device in some implementations. In other implementations, the third electrode 356 is included in the device. The wearable device can be implemented with four electrodes, with five electrodes, or with more or fewer electrodes to capture the desired physiological and other parameters. Referring again to FIG. 3, the SpO2 circuitry 338 and the ECG and respiration circuitry 340 include electrodes for sensing the physiological parameters. The sensed signals corresponding to the physiological parameters are collected in the SpO2 circuitry 338 and the ECG and respiration circuitry 340. In some implementations, the signals can be detected as analog data and converted to a digital signal within the SpO2 circuitry 338 and the ECG and respiration circuitry 340 before the data is transmitted to the microcontroller 336. In some implementations, the signals can be detected as analog data by the SpO2 circuitry 338 and the ECG and respiration circuitry 340 and then is transmitted to and converted into digital data by the microcontroller 336. In some implementations, the SpO2 circuitry 338 and the ECG and respiration circuitry 340 include processing components for filtering and conditioning of the sensed signals before transmitting the signals to the microcontroller 336. Filtering the signals can remove artifacts from the signal including artifacts inserted into the signal by motion of the user or by power line interference. The processing components of the SpO2 circuitry 338 and the ECG and respiration circuitry 340 can include hardware and/or software.

After the signals are processed to remove artifacts, the SpO2 circuitry 338 and the ECG and respiration circuitry 340 transmit the signals to the microcontroller 336. In some implementations, the microcontroller 336 includes processing components for further conditioning of the signals to remove noise and artifacts. In some implementations, the microcontroller 336 performs the conditioning to remove motion and power line interference artifacts rather than the SpO2 circuitry 338 and the ECG and respiration circuitry 340. After the signals have been processed, the microcontroller 336 transmits the signal data to an external client device via the antenna 342. In some implementations, additionally or alternatively, the conditioning of the signal is completed at the client device. The antenna 342 transmits the data from the microcontroller 336 to an external device such as a smartphone over a wireless communication channel using WiFi, Bluetooth, radio transmissions, and/or any other suitable communication means. In some implementations, the microcontroller 336 transmits the data through the antenna 342 intermittently. Intermittent transmission of the data to a client device can allow additional sensor devices to communicate with the client device to provide additional physiological or other information to the client device. In some implementations, the microcontroller 336 transmits the data through the antenna 342 continuously.

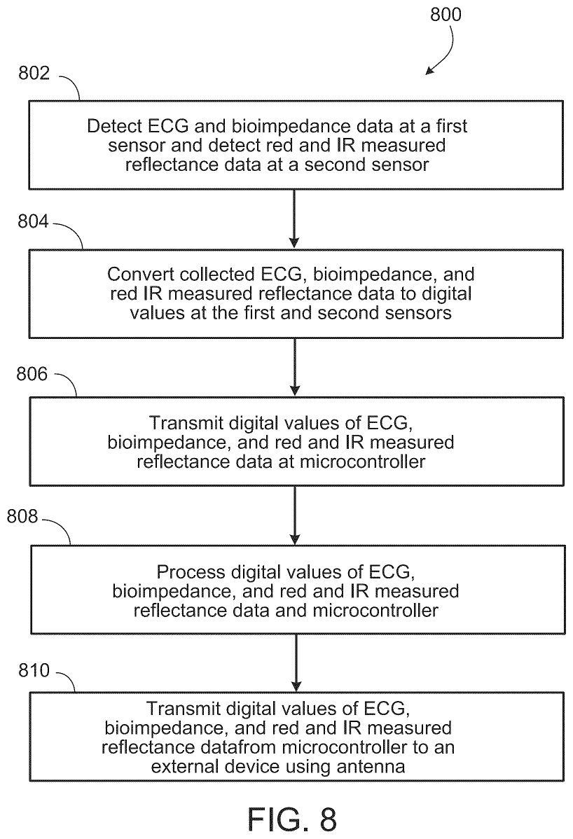

For example, FIG. 8 illustrates the method 800 of monitoring physiological parameters associated with seizure activity, the cardiorespiratory dysfunctions, and/or associated with SUDEP at the wearable device (e.g., wearable device 100 of FIGS. 1A-1C, wearable device 220 of FIG. 2, and wearable device 300 in FIG. 3) and processing the data using components of the wearable device described in FIG. 3. At step 802, ECG and bioimpedance data are detected at a first sensor 340 and red and IR measured reflectance values are detected at a second sensor 338. The red and IR measured reflectance values can be converted to an SpO2 value. The conversion of the red and IR measured reflectance values to an SpO2 value can occur on the wearable device or on an external device, as will be described in greater detail below. At step 804, the collected ECG and bioimpedance data, and red and IR measured reflectance values are converted to digital values at the first sensor 340 and the second sensor 338. At step 806, digital values of ECG, bioimpedance, and SpO2 data are transmitted to the microcontroller 336. At step 808, digital values of ECG and bioimpedance data, and red and IR measured reflectance values are processed at the microcontroller 336. At step 810, the digital values of the ECG, bioimpedance, and red and IR measured reflectance values are transmitted from the microcontroller 336 to an external device using the antenna 342.

Referring again to FIG. 3, the power management module 346 is coupled to a battery 344 and can provide power to the microcontroller 336, the SpO2 circuitry 338, and the ECG and respiration circuitry 340 through the voltage regulator 348. The power management module 346 provides an indication of battery charge, battery and device status, and power reserves through a visual indicator such as one or more LED lights on the wearable device. In some implementations, the battery is a rechargeable battery. In some implementations, the battery is rechargeable by the use of a wired connection, such as a micro-USB connected to the wearable device at the data/power connector 350. In some implementations, the battery can be charged wirelessly, for example by the use of a wireless charging coil.

The multi-modal input to the wearable device provides sensed physiological data including cardiac and respiratory information that can be used to assess whether a seizure has occurred, whether any cardiorespiratory dysfunctions have occurred, and/or whether a seizure, cardiorespiratory dysfunction, or SUDEP event is likely to occur. In some implementations, the device is positioned over the carotid artery on the wearer's neck. The positioning of the device sensors on the user's neck and over the carotid artery provides suitable signal integrity while enabling measurement of SpO2, respiration, and cardiac activity (ECG). In other implementations, the device need not be positioned such that the device sensors are above the carotid artery, and the device can instead be positioned elsewhere on the front of the neck, back of the neck, or at other positions on the body including the chest. In some implementations, the wearable device is positioned near an artery in the neck of the wearer, for example near the carotid artery or near the sternocleidomastoideole. In some implementations, the positioning of the device can be dependent on the physiological parameters and conditions that the device is intended to monitor. For example, the device can be positioned in a different location when used for monitoring sleep patterns or sleep apnea. In another example, the device can be positioned in a different location when used for monitoring for cardiorespiratory dysfunction and/or SIDS likelihood in an infant. For example, in some implementations the device can be sized and configured to be positioned on one of an arm, a leg, a head, or a back of a user.

In some implementations, the data/power connector 350 enables one or both of charging (e.g., via micro-USB or similar) and data transmission in or out of the device. For example, the data/power connector 350 can allow the wearable device to be coupled to an external device such as a computer to transmit historical data stored in the wearable device to the computer. In another example, the data/power connector 350 can allow the wearable device to be coupled to an external device to transmit software updates from an external device to the microcontroller 336, SpO2 circuitry 338, ECG and respiration circuitry 340, or other components of the wearable device. This connection can also allow the wearable device to be reset or upgraded. In some implementations, the wearable device does not include a data/power connector 350, and all charging and data transmission is accomplished wirelessly. In such implementations, the wearable device can be sealed with no openings between the interior circuitry and external environment. The wearable device can be waterproof or water resistant. In some implementations, the microcontroller 336 of the wearable device is capable of transmitting and/or receiving data to/from a second microcontroller located in a second wearable device of a user. In some implementations, the microcontroller 336 is capable of communication with the second wearable device after pairing of the devices to associate the devices with one another. In some implementations, the microcontroller can transmit signals to direct a monitoring behavior of the second wearable device, or can be directed to alter a monitoring behavior based on received instructions from the second wearable device.

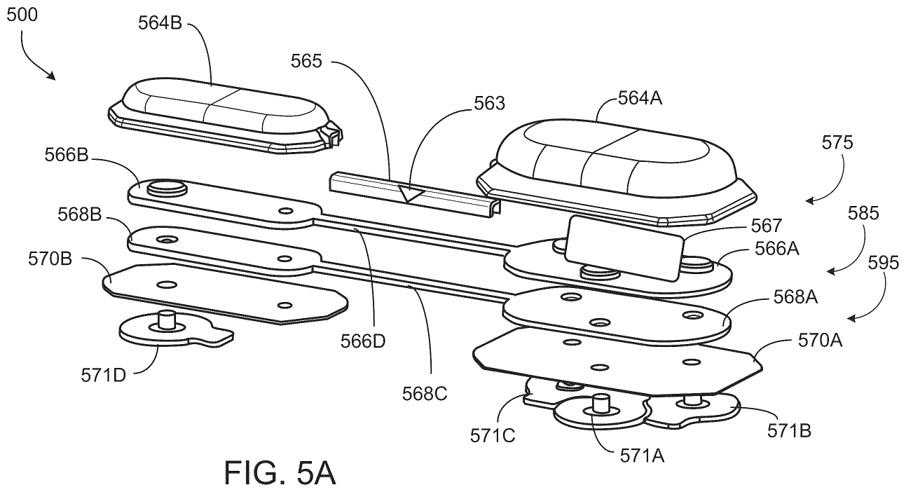

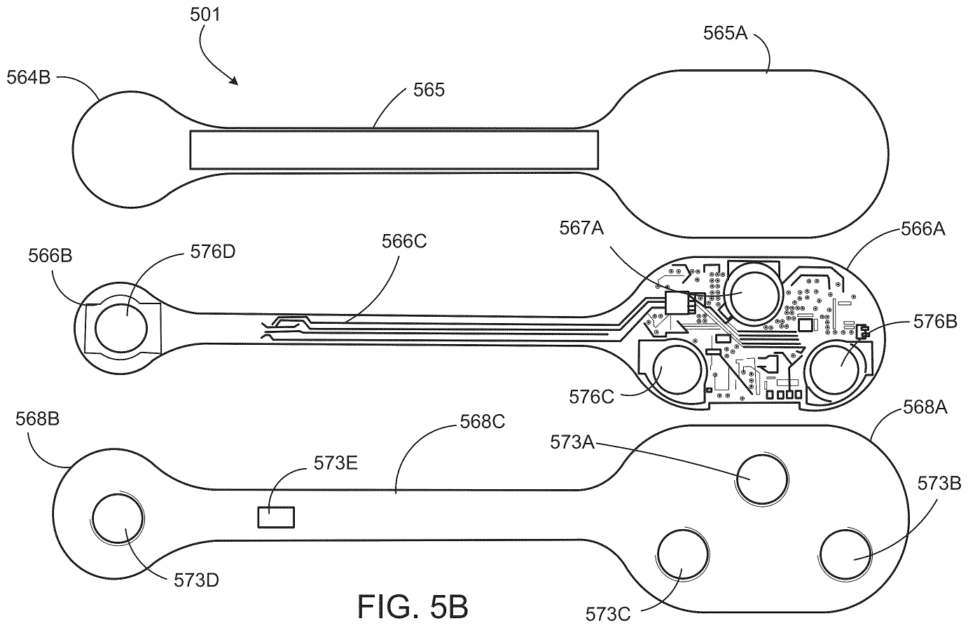

FIGS. 5A and 5B illustrate views of layers in an exemplary wearable device 500 (e.g., wearable device 100 of FIGS. 1A-1C, wearable device 220 of FIG. 2, and wearable device 300 in FIG. 3). FIG. 5A shows an exploded view of the layers of the wearable device 500, and FIG. 5B shows some of the layers side by side. The wearable device 500 includes a top housing layer 575 including a first outer housing 564A, second outer housing 564B and bridge portion 565. Within the first outer housing 564A, second outer housing 564B and bridge portion 565 is a printed circuit board (PCB) layer 585 including a first node 566A, second node 566B and bridge portion 566C connecting the first node 566A to the second node 566B. Below the PCB layer is a layer 595 (for example, a silicone layer or layer of other suitable material) forming a bottom of an enclosure portion of the wearable device. The layer 595 includes a first node 568A, a second node 568B, and a bridge portion 568C.

As described above with respect to FIGS. 1A-1C, the wearable device is positioned with a bridge portion 565 extending between two outer housings 564A and 564B which provide protection to underlying components which are in communication with a first electrode 571A, second electrode 571B, third electrode 571C, and fourth electrode 571D for sensing physiological parameters at the skin of the user. In some implementations, a fifth electrode (not shown) is positioned on a back of the wearable device for use in detection of values for SpO2 measurement. The wearable device 500 is attached to the user's skin at the neck using a first adhesive patch 570A and a second adhesive patch 570B shaped to correspond to the bottom surface of the enclosure portion of the wearable device 500, and made from materials that do not irritate the skin at the neck. In some implementations, the adhesive is a silicone-based adhesive. In some implementations, the bridge section 565 of the wearable device 500 includes an alignment marker 563 on a top surface which can be visually aligned with the midline of the neck to provide correct positioning of the electrodes on the user's neck. In some implementations, the user may have a permanent or semi-permanent tattoo or other marking at a midline of the neck to be aligned with the alignment marker 563 to ensure appropriate positioning of the wearable device 500.

The first outer housing 564A, second outer housing 564B and bridge portion 565 forming the top housing layer 575 are formed of a polymer material to provide flexible protection of the underlying layers and components. The first outer housing 564A, second outer housing 564B and bridge portion 565 can be formed as a modular housing or as a single housing component which contains the circuitry, components, and power source. The top housing layer 575 including first outer housing 564A and second outer housing 564B can be formed from a material that provides a rigid housing for the underlying components, while the bridge portion 565 of the top housing layer 575 is flexible. In some implementations, the first outer housing 564A and the second outer housing 564B are formed from a different material than the bridge portion 565.

The bridge portions 565, 566C, and 568C are formed as narrow regions coupling wider ends (“nodes”) of each layer. The narrow bridge portions 565, 566C, and 568C improve the flexibility of the wearable device 500 at the center portion to allow the wearable device 500 to be used on a variety of user neck sizes and shapes. Additionally, the flexibility in the center portion of the wearable device 500 improves comfort during use, as this portion rests over the front portion of the throat near the vocal cords and laryngeal prominence of thyroid cartilage. The bridge portions 565, 566C, and 568C allow the center portion to rest comfortably over the throat to allow speaking, swallowing, and breathing with minimal discomfort or chafing. In some implementations, the bridge portions 565, 566C, and 568C are wider or narrower than the illustration in FIG. 5A. In some implementations, the width of the bridge portions 565, 566C, and 568C is the same as the end portions or nodes coupled by the bridge.

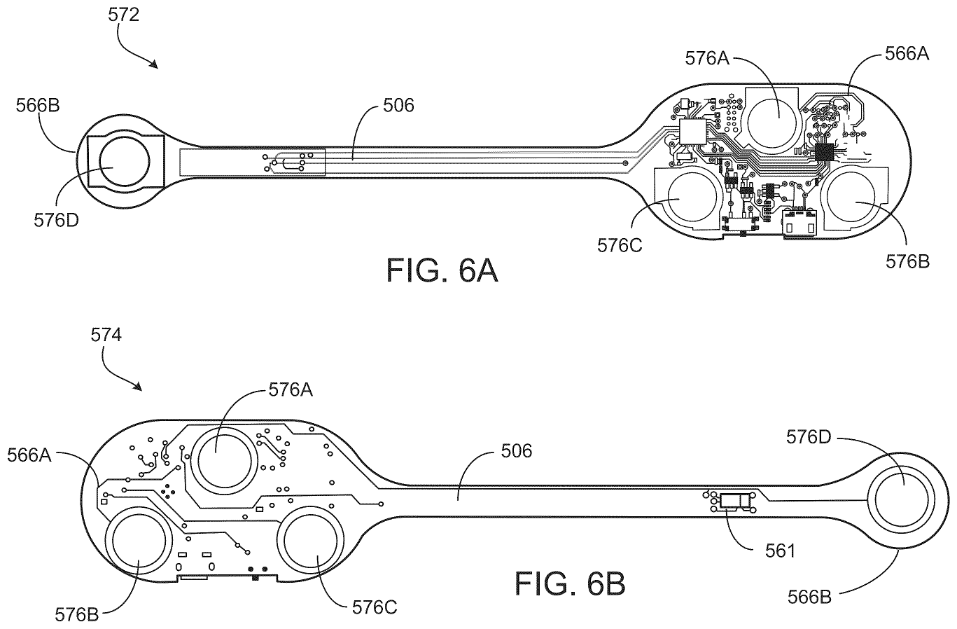

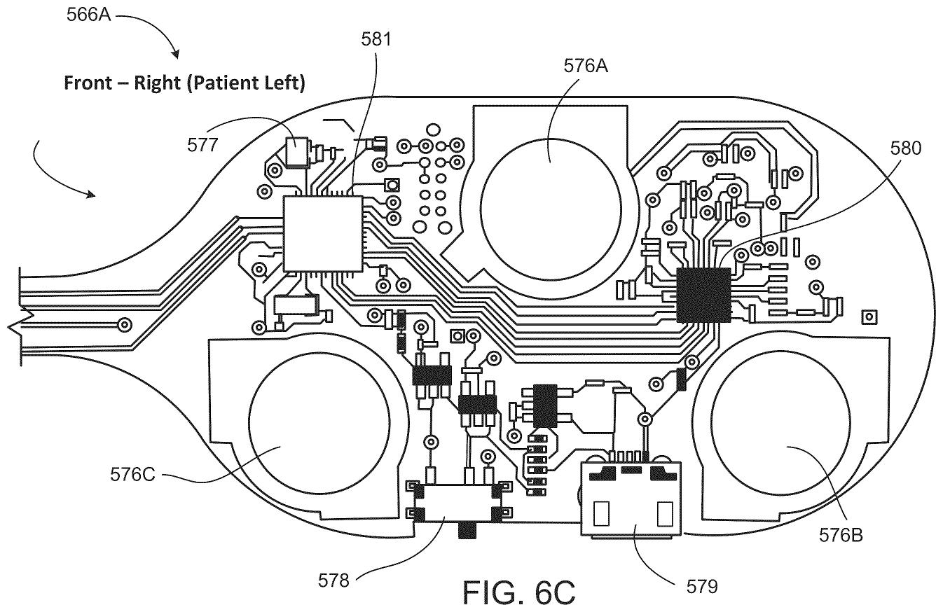

The first node 566A, second node 566B and bridge portion 566C is formed as a flexboard layer or printed circuit board (PCB) layer 585 including required circuitry and a battery or power source 567. The first node 566A includes a first aperture 576A, second aperture 576B, and third aperture 576C formed through the PCB layer 585. The second node 566B includes a fourth aperture 576D through the PCB layer 585. The first aperture 576A, second aperture 576B, third aperture 576C, and fourth aperture 576D allow the first electrode 571A, second electrode 571B, third electrode 571C, and fourth electrode 571D electrodes to extend through the wearable device 500 to the circuitry of the PCB layer 585. The back side of the bridge portion 566C includes sensor 561 (shown in FIG. 6B). In some implementations, sensor 561 includes LEDs and photodetectors and is associated with the SpO2 sensing.

The layer 595 also includes first aperture 573A, second aperture 573B, and third aperture 573C formed in the first node 568A, and a fourth aperture 573D and a fifth aperture 573E formed in the second node 568B to permit the electrodes, as well as LED lights and photodiodes, to extend through the layer 595 from contact with the user's skin to the circuitry at the PCB layer 585. The fifth aperture 573E allows the sensor circuitry 561 to extend through the layer 595 to the user's skin. In some implementations, a female snap connector is positioned within the first aperture 576A, second aperture 576B, third aperture 576C, and fourth aperture 576D of the PCB layer 585 to allow the electrodes 571A-571D to be connected to the wearable device 500.

The printed circuit board layer 585 is illustrated in FIG. 6A-6C. FIG. 6A shows the front view 572 of the printed circuit board layer 585, FIG. 6B shows the back view 574 of the printed circuit board layer 585, and FIG. 6C shows an expanded view of the first node 566A of the printed circuit board layer 585 according to the front view 572 shown in FIG. 6A. The front view 572 of the printed circuit board layer 585, shown in FIG. 6A, includes a first node 566A and a second node 566B. The first node 566A (shown in greater detail in FIG. 6C) includes a first aperture 576A, second aperture 576B, and third aperture 576C. The second node 566B is coupled to the first node 566A by a bridge portion 506. The second node 566B includes a fourth aperture 576D. The back view 574 of the printed circuit board layer 585, shown in FIG. 6B, includes the first aperture 576A, second aperture 576B, and third aperture 576C formed in the first node 566A and the fourth aperture 576D formed in the second node 566B. The first aperture 576A, second aperture 576B, third aperture 576C, and fourth aperture 576D allow electrodes to extend through the printed circuit board layer 585 to interact with the circuitry of the ECG and respiration sensors on the front view 572. A photo plethysmography (PPG) sensor 561 for SpO2 sensing including LEDs and photodetectors is located on the backside of the printed circuit board layer 585, positioned to receive light reflected from a user's skin when the back side of the printed circuit board layer 585 is oriented toward the user's neck. In some implementations, a pressure sensor (not shown) is located on the backside of the printed circuit board layer 585. The positioning of the majority of the components and circuitry on the front side of the printed circuit board layer 585 allows the wearable device 500 to be flat on the bottom side so that the wearable device 500 is conformal to and flush with the skin of the user's neck. The positioning of the wearable device 500 flush against the skin of the user's neck improves the accuracy of the sensed physiological parameters by eliminating artifacts arising from relative motion between the user's neck and the device. Additionally, the positioning of the wearable device 500 flush against the skin of the user's neck improves user comfort during use of the wearable device 500.

As illustrated in FIG. 6C, the first node 566A on the front view 572 also includes a microcontroller 581 (including one or more processors and memory with instructions thereon) and an oscillating crystal 577 for use with the microcontroller 581. First node 566A also includes a data/power connector 579 and an indicator 578. In some implementations, the data/power connector 579 is a USB connector for charging a battery. In some implementations, the data/power connector 579 is a USB connector for transmitting and/or receiving data to/from an external device. In some implementations, data/power connector 579 is a wireless charging coil for wirelessly charging a battery. In some implementations, the indicator 578 is an on/off switch for controlling power and operation of the wearable device. In some implementations, the indicator 578 is an LED indicator which indicates to a user a status of the wearable device 500, such as a status of the battery charge, an on/off status of the wearable device 500, or a transmission mode of the wearable device 500.

A first adhesive patch 570A is positioned on a bottom surface of the first node 568A of the layer 595 opposite the first outer housing 564A. A second adhesive patch 570B is positioned on a bottom surface of the second node 568B of the layer 595 opposite the second outer housing 564B. The first adhesive patch 570A and the second adhesive patch 570B are shaped to match a similar shape of the bottom surface of the first node 568A and the second node 568B of the layer 595. In some implementations, the first adhesive patch 570A and the second adhesive patch 570B are shaped to have a larger or smaller width and/or length than the first node 568A and the second node 568B of the layer 595. In some implementations, the first adhesive patch 570A and the second adhesive patch 570B are formed from reusable medical-grade adhesives. The use of medical-grade adhesives can prevent irritation of the skin at the user's neck with use of the wearable device 500. In some implementations, the first adhesive patch 570A and the second adhesive patch 570B are formed from single-use adhesive patches that can be replaced for each use of the wearable device 500. The use of two adhesive patches, rather than a single adhesive patch underlying the entire wearable device 500 provides a conformal fit of the wearable device 500 to the neck of the user and allows movement of the skin beneath the wearable device 500 at the throat.

The first adhesive patch 570A and the second adhesive patch 570B allow the first electrode 571A, second electrode 571B, third electrode 571C, and fourth electrode 571D to sense physiological parameter signals at the skin for acquisition at the circuitry in the PCB layer 585. In some implementations, the electrodes are incorporated into the adhesive patches 570A and 570B. In some implementations, additional electrodes are included with the first adhesive patch 570A and the second adhesive patch 570B. In some implementations, the first electrode 571A, second electrode 571B, third electrode 571C, and fourth electrode 571D each are accessible at or through the bottom surface of the first node 568A and the second node 568B of the layer 595, and the first adhesive patch 570A and the second adhesive patch 570B are formed with apertures through the adhesive material so that the first electrode 571A, second electrode 571B, third electrode 571C, and fourth electrode 571D are in contact with the skin of the user's neck through the adhesive patches 570A and 570B. In some implementations, the first electrode 571A, second electrode 571B, third electrode 571C, and fourth electrode 571D are formed from single-use electrodes that can be replaced for each use of the wearable device 500.

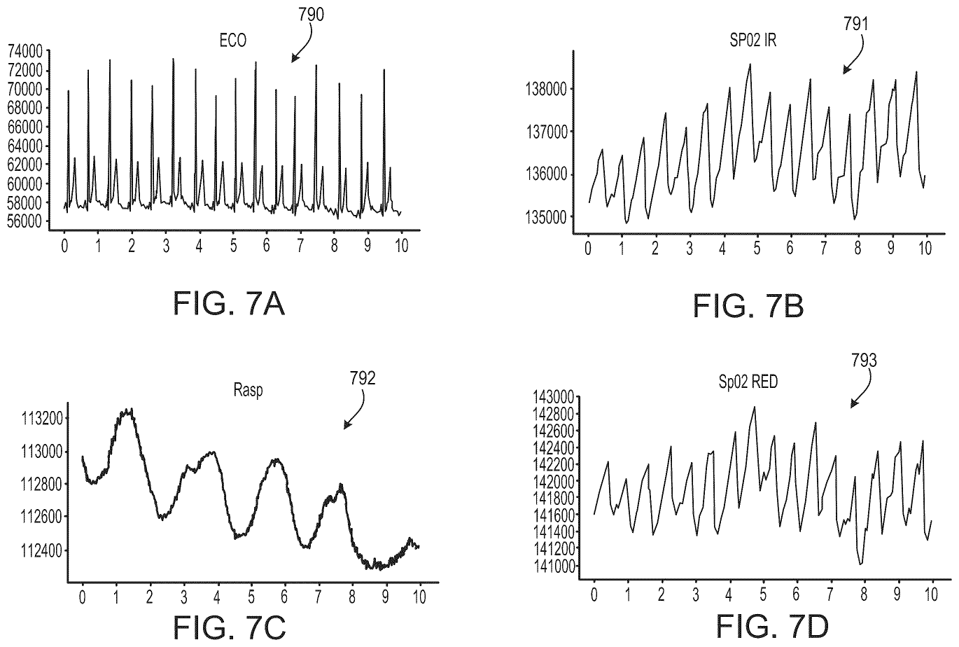

FIGS. 7A-7D illustrate graphical representations of signals detected concurrently at a wearable device (e.g., wearable device 100 of FIGS. 1A-1C, wearable device 220 of FIG. 2, wearable device 300 in FIG. 3, and wearable device 500 in FIGS. 5A-5C). As described above, the wearable device includes circuitry to sense ECG signals, SpO2 data, and respiration patterns. The graphical representations of the data detected at the sensors includes a graph of the detected ECG data 790 (FIG. 7A), a graph of detected reflected light in the infrared spectrum 791 for SpO2 calculation (FIG. 7B), a graph of detected reflected light in the red spectrum 793 for SpO2 calculation (FIG. 7D), and a graph of detected respiration patterns 792 (FIG. 7C). The information displayed in the graph of detected reflected light in the infrared spectrum 791 (FIG. 7B) and the graph of detected reflected light in the red spectrum 793 (FIG. 7D) is used to calculate the oxygen saturation, or SpO2, of the blood. This calculation can occur in the wearable device, in the client device, or in the external processor.

Although the graphical representations of sensed physiological parameters are shown separately in FIGS. 7A-7D, the circuitry and components of the wearable device described above can sense and monitor the parameters simultaneously. Further the sensed data can be displayed as separate graphical representations as depicted here, or on a same graph or timeline as necessary. The information illustrated in the graphical representations is also analyzed by an algorithm or program to detect changes, events, and patterns indicative of seizure activity, cardiorespiratory dysfunctions, or other events based on predetermined settings and thresholds, machine learning training sets, or other determination methods. In some implementations, the algorithm or program detects these changes, events, and patterns by analyzing the sensed data to determine various metrics of the signals, including but not limited to respiration rate, tidal volume, heart rate, heart rate variability, and timings between waves of the signal (for example, the time between Q and T wave in ECG signal). Additionally, the algorithm or program can assess the detected changes, events and patterns to determine and output a likelihood of SUDEP event based on predetermined settings and thresholds, machine learning training sets, or other determination methods.

In some implementations, the data detected at the sensors is displayed at the client device, in a physician or patient portal, or on an external device of a caregiver in a graphical format as illustrated in FIGS. 7A-7D. In some implementations, the data is displayed in an application or program running on the client device, physician or patient portal, or external device.

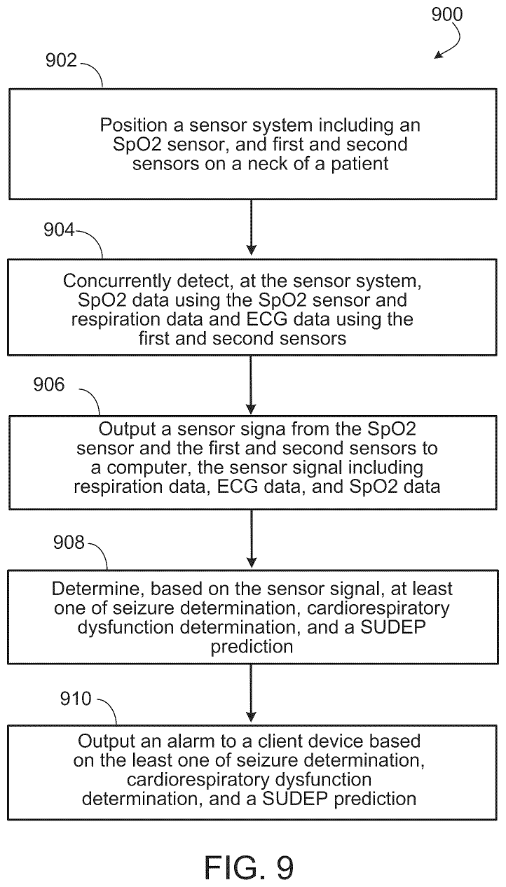

FIG. 9 illustrates an exemplary flow chart for a method 900 of detecting physiological parameters related to seizure activity and cardiorespiratory dysfunctions, and using the detected parameters for predicting SUDEP (such as predicting the occurrence of SUDEP, predicting the likelihood of SUDEP, a near-SUDEP/SUDEP forecast, and/or predicting the timing of SUDEP). Method 900 can be performed in a system (e.g., system 200 in FIG. 2) including a wearable device (e.g., wearable device 100 of FIGS. 1A-1C, wearable device 220 of FIG. 2, and wearable device 300 in FIG. 3). At step 902, a sensor system including a SpO2 sensor and first and second sensors are positioned on a neck of a patient. At step 904, the sensor system concurrently detects SpO2 data using the SpO2 sensor and detects respiration data and ECG data using the first and second sensors. At step 906, a sensor signal is output from the SpO2 sensor and the first and second sensors to a computer (e.g., a processor, mobile device, smart phone, desktop or laptop computer, cloud-based device, or other external processing device). The sensor signal includes respiration data, ECG data, and SpO2 data. At step 908, a seizure determination, a cardiorespiratory dysfunction determination, and/or a SUDEP determination (such as a prediction of SUDEP, a prediction of the likelihood of SUDEP, or a prediction of a time of SUDEP) is output from the computer based on the sensor signal. At step 910, an alarm is output to a client device based on the seizure determination, the cardiorespiratory dysfunction determination, and/or the SUDEP determination. Alternatively or in addition, at step 910 an alarm can be output to a caregiver device and/or a patient or physician portal.

In a first example, a system (e.g., system 200 in FIG. 2) including a wearable device (e.g., wearable device 100 of FIGS. 1A-1C, wearable device 220 of FIG. 2, and wearable device 300 in FIG. 3) can be used to predict SUDEP based at least in part on detecting one or more seizures and/or cardiorespiratory dysfunction. The system can generate and output a SUDEP determination (such as predicting the occurrence of SUDEP, predicting the likelihood of SUDEP, and/or predicting the timing of SUDEP) as a function of sensed factors including a type, timing, and/or quantity of seizures detected and/or type, timing, and/or quantity of cardiorespiratory dysfunctions detected.

In a second example, a system (e.g., system 200 in FIG. 2) including a wearable device (e.g., wearable device 100 of FIGS. 1A-1C, wearable device 220 of FIG. 2, and wearable device 300 in FIG. 3) can be used to predict SUDEP based at least in part on detecting physiological parameters, such as cardiorespiratory dysfunctions, occurring before a seizure, during a seizure, and/or after a seizure. The system can generate and output a SUDEP determination (such as predicting the occurrence of SUDEP, predicting the likelihood of SUDEP, and/or predicting the timing of SUDEP) as a function of sensed factors including physiological parameters occurring before a seizure, during a seizure, or after a seizure.

In a third example, a system (e.g., system 200 in FIG. 2) including a wearable device (e.g., wearable device 100 of FIGS. 1A-1C, wearable device 220 of FIG. 2, and wearable device 300 in FIG. 3) can be used to predict SUDEP based at least in part on detecting physiological parameters, such as cardiorespiratory dysfunctions, occurring after a seizure. The system can generate and output a SUDEP determination (such as predicting the occurrence of SUDEP, predicting the likelihood of SUDEP, and/or predicting the timing of SUDEP) by sensing physiological parameters that occur after a seizure and determining that the sensed physiological parameters are of the type that occur in people who suffer SUDEP after a seizure.

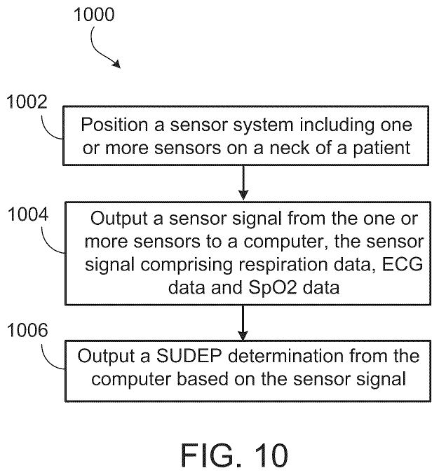

FIG. 10 illustrates an exemplary flow chart for a method 1000 of predicting, seizure-related death at a wearable device (e.g., wearable device 100 of FIGS. 1A-1C, wearable device 220 of FIG. 2, and wearable device 300 in FIG. 3). The wearable device monitors physiological parameters, such as seizure activity or cardiorespiratory dysfunctions, of a user which are used to determine a likelihood or risk of SUDEP. In step 1002, a sensor system is positioned on the neck of a patient. The sensor system includes one or more sensors. The one or more sensors are used to detect physiological signals, such as the respiration data, ECG data, and SpO2 data.

At step 1004, a sensor signal is output from the one or more sensors to an external device, such as a smartphone, computer, or other external computing device. The sensor signal includes at least respiration data, ECG data, and SpO2 data. In some implementations, the sensor signal includes data related to one or more additional physiological parameters. For example, the sensor signal can include data related to a skin temperature of a user, motion of a user, or other parameters. At step 1006, a SUDEP determination (such as a prediction of SUDEP, a prediction of the likelihood of SUDEP, or a prediction of a time of SUDEP) is output from the computer. The SUDEP determination is based on an analysis of the sensor signal. The analysis can be conducted in software or an algorithm located at the computer or smartphone, or in an external processor or cloud-based server. In some implementations, the analysis is based on a set threshold or data pattern. In some implementations, the analysis is based on a machine learning or artificial intelligence system trained on clinical data related to SUDEP, seizure activity, and cardiorespiratory activity. In some implementations, a seizure event determination (such as anticipated seizure activity, seizure activity occurrence, or a near-SUDEP event) and/or a cardiorespiratory dysfunction event determination is output from the computer in addition to or instead of the SUDEP determination. In some implementations, the seizure event determination includes a categorization or details related to the seizure event detected. The seizure event determination is based on analysis of the sensor signal. In some implementations, the cardiorespiratory dysfunction event determination includes a categorization or details related to the cardiorespiratory dysfunction event detected. The cardiorespiratory dysfunction event determination is based on analysis of the sensor signal.

In some implementations, the method also includes combining the sensor signal from the sensor system with data from a second system located on a body part of the patient. For example, the second system can be located on a patient's arm, finger, wrist, leg, chest, or any other suitable position. The combination of the sensor signal with data from the second system can occur prior to the outputting of the SUDEP determination from the computer, such that the SUDEP determination is based on the sensor signal and the data from the second system. In some implementations, the method also includes filtering the sensor signal to remove motion artifacts. In some implementations, the method includes processing or filtering the sensor signal to remove noise from the data.

In some implementations, the method includes outputting an alarm to a client device based on the SUDEP determination. For example, when the external device, such as a computer or smartphone, determines from the sensor signal that a SUDEP event is likely (such as likely in an immediate future or a less immediate future), the external device can output an audible, visual, or text-based alarm to a specified client device to warn a caregiver of the likely SUDEP event. The external device can also output an alarm to a physician or patient portal, a call center, or an emergency department when a SUDEP event is determined to be likely to occur.

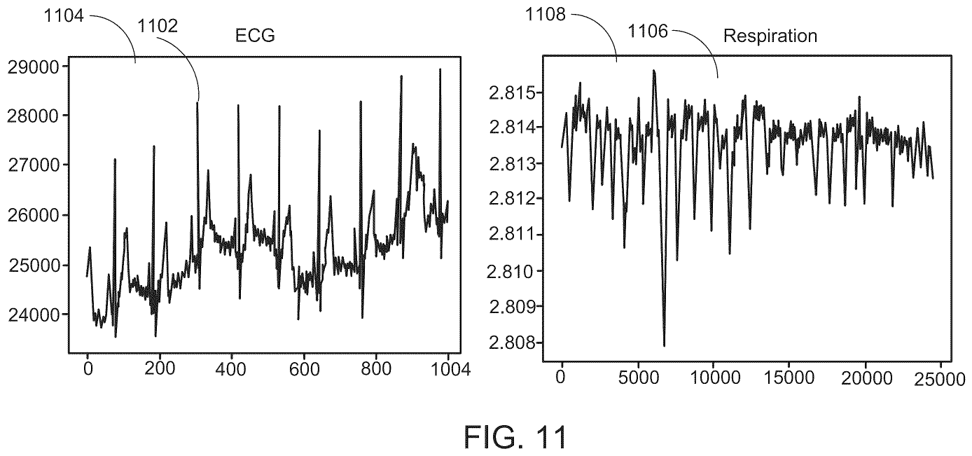

FIG. 11 illustrates experimental data 1100 recording sensor outputs from a wearable device (e.g., wearable device 100 of FIGS. 1A-1C, wearable device 220 of FIG. 2, and wearable device 300 in FIG. 3). The experimental data 1100 includes an ECG signal 1104 showing an ECG trace 1102 over a period of time as detected and recorded by ECG circuitry of the wearable device at the neck of a user. Additionally, the experimental data 1100 includes a respiration signal 1108 showing a trace 1106 of a respiration pattern of a user over a different period of time. In both the ECG signal 1104 and the respiration signal 1108, the x-axis represents the sample number and the y-axis corresponds to a digital value based on the analog voltage of the signal. As described above, the respiration and ECG data are monitored at a common circuitry component using common electrodes for simultaneously sensing the two physiological parameters.

The wearable device includes sensors for determining user physiological parameters from which seizure activity, cardiorespiratory dysfunctions, and likelihood of SUDEP events can be determined. The wearable device is capable of monitoring multiple physiological parameters using a limited number of sensors to maintain a small form factor that can be comfortably worn by a user at the user's neck. The wearable device also includes a flexible bridge portion between the two sensor nodes so that the sensor nodes can be positioned at the carotid artery on either side of the neck, while the flexibility of the wearable device at the central portion of the neck allows the user to breathe, talk, and swallow normally without discomfort or chafing. The wearable device can further include alignment markers to aid in the positioning of the device for accurate detection of physiological parameters, and can be used with single-use or reusable adhesive patches and electrodes that can minimize or reduce irritation at the user's neck. The information collected by the sensors of the wearable device can be used at the wearable device, a client device, or an external processor or cloud-based server, to determine seizure activity, cardiorespiratory dysfunctions, and SUDEP events and caregivers or emergency personnel can monitor the data and receive a warning when a likelihood of SUDEP is determined.

In other embodiments, a system (e.g., system 200 in FIG. 2) including a wearable device (e.g., wearable device 100 of FIGS. 1A-1C, wearable device 220 of FIG. 2, and wearable device 300 in FIG. 3) can be used to predict other events based at least in part on detecting physiological parameters. In one example, the system including the wearable device can be used to predict sudden infant death syndrome (SIDS) based at least in part on detecting physiological parameters. In another example, the system including the wearable device can be used to detect or predict sleep apnea or sleep-apnea related events based at least in part on detecting physiological parameters.

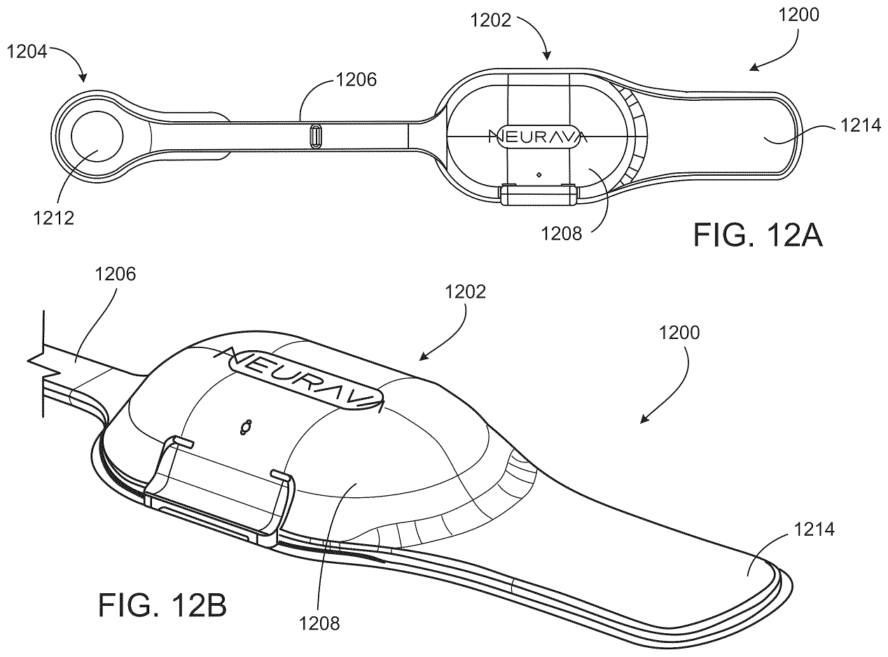

Referring to FIGS. 12A and 12B, an example embodiment of a wearable device 1200 includes a first node 1202 and a second node 1204. The first node 1202 includes a first outer housing 1208 and the second node 1204 includes a second outer housing 1212. The first outer housing 1208 and the second outer housing 1212 are coupled by a bridge portion 1206. The wearable device 1200 can have one, more than one, or all features described above.

The wearable device 1200 also includes a securement portion 1214. The securement portion 1214 can be a flap that extends from a portion of the wearable device 1200. For example, in the illustrated embodiment the securement portion 1214 extends from an end of the first node 1202. The securement portion 1214 can include an adhesive to assist in holding the wearable device 1200 in place on the skin of a user. The securement portion 1214 can be beneficial, for example, to hold the wearable device 1200 on body parts of different sizes, such as on smaller necks. In some embodiments, the wearable device 1200 can include more than one securement portion 1214. In some embodiments, the one or more securement portions 1214 can extend from the wearable device 1200 in other directions and/or orientations.

A number of embodiments of the invention have been described. Nevertheless, it will be understood that various modifications may be made without departing from the scope of the invention. For example, size, shape, and orientation of various components of the wearable devices can be modified as appropriate for a given application. Similarly, one or more components described with respect to one example of wearable devices can be combined with other examples of wearable devices described herein. Accordingly, other embodiments are within the scope of the following claims.

1. A system comprising:
an ECG sensor;
an SpO2 sensor;
a respiration sensor; and
a wearable housing sized and configured to be worn on one of a neck, an arm, a leg, a head, a back, or a chest of a user, wherein the ECG sensor, the SpO2 sensor, and the respiration sensor are positioned in the wearable housing at locations configured to sense physiological parameters of the user.

2. The system of claim 1, and further comprising:
a first circuitry in data communication with the SpO2 sensor; and
a second circuitry in data communication with at least one of the ECG sensor and the respiration sensor.

3. The system of claim 2, wherein the second circuitry is in data communication with both of the ECG sensor and the respiration sensor.

4. The system of claim 2, and further comprising:
a microcontroller in data communication with both the first circuitry and the second circuitry; and
at least one antenna connected to the microcontroller and configured to transmit data to an external device and receive data from the external device.

5. (canceled)

6. The system of claim 4, wherein the microcontroller is configured to:
receive data from the first circuitry and the second circuitry;
process the data from the first circuitry and the second circuitry;
determine an occurrence of a seizure event or a cardiorespiratory dysfunction based on the data received from the first circuitry and the second circuitry; and
transmit the processed data to the external device via the antenna.

7. The system of claim 4, wherein the microcontroller is configured to:
receive data from the first circuitry and the second circuitry;
process the data from the first circuitry and the second circuitry; and
intermittently transmit the processed data to the external device via the antenna based on a preprogrammed schedule, and
wherein the external device is configured to determine an occurrence of a seizure event or a cardiorespiratory dysfunction based on the data received from the first circuitry and the second circuitry.

8. The system of claim 4, wherein the microcontroller is further configured to:
transmit and receive data from an external microcontroller, the external microcontroller associated with a second wearable device.

9. (canceled)

10. (canceled)

11. (canceled)

12. The system of claim 6, wherein the processed data is configured to be used to detect a likelihood of SUDEP.

13. The system of claim 1, wherein the wearable housing is positioned on a front portion of the neck of the user.

14. The system of claim 13, wherein the wearable housing comprises a first housing and a second housing, the first housing including a first sensor comprising the ECG sensor and the second housing including a second sensor comprising the respiration sensor, such that the first and second sensors are mounted in the wearable housing at locations on opposite sides of the neck of the user, and the wearable housing further comprises a third sensor mounted in the wearable housing and configured to provide a right leg drive to improve signal quality.

15. The system of claim 14, wherein the SpO2 sensor comprises a fourth sensor mounted in the wearable housing at a location positioned near an artery in the neck of the user.

16. (canceled)

17. (canceled)

18. (canceled)

19. The system of claim 13, further comprising means for attaching the system to the neck of the user.

20. (canceled)

21. (canceled)

22. The system of claim 1, further comprising:
a rechargeable battery coupled to the ECG sensor, the SpO2 sensor, and the respiration sensor, and configured to provide power to the ECG sensor, the SpO2 sensor, and the respiration sensor, and
at least one of USB power input and a wireless charging coil for charging the rechargeable battery.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. The system of claim 6, wherein the microcontroller is configured to intermittently transmit the processed data to the external device based on a preprogrammed schedule.

58. (canceled)

59. (canceled)

60. (canceled)

61. (canceled)

62. The system of claim 7, wherein the processed data is configured to be used to detect a likelihood of SUDEP.

63. (canceled)

64. The system of claim 1, wherein the ECG sensor and the respiration sensor comprise first and second sensors mounted in the wearable housing at locations on the arm or leg of the user, the wearable housing further comprising a third sensor mounted in the wearable housing and configured to provide a right leg drive and wherein the SpO2 sensor comprises a fourth sensor mounted in the wearable housing at a location positioned near an artery in the arm or the leg of the user when the wearable housing is positioned on the arm or the leg of the user.

65. (canceled)

66. The system of claim 14, wherein the wearable housing further includes:
a flexible bridge extending between the first housing and the second housing, the first housing positioned at a first end of the flexible bridge and the second housing positioned at a second end of the flexible bridge.

67. (canceled)

68. (canceled)

69. (canceled)

70. (canceled)

71. (canceled)

72. (canceled)

73. (canceled)

74. (canceled)

75. The system of claim 6, wherein the microcontroller is further configured to:
output an alarm to a client device based on the determination of an occurrence of a seizure event or a cardiorespiratory dysfunction.

76. The system of claim 7, wherein the external device is further configured to:
output an alarm to a client device based on the determination of an occurrence of a seizure event or a cardiorespiratory dysfunction.

77. The system of claim 14, wherein the first housing and the second housing are configured to be communicatively coupled.
